(19) United States
(12) Patent Application Publication
(10) Pub. No.: US 2008/0182774 A1
(43) Pub. Date: Jul. 31, 2008

(54) MIXTURES CONTAINING 3-(4-METHYLCYCLOHEX-3-ENYL)BUTYRALDEHYDE AND 2,6-DIMETHYL-7-OCTEN-2-OL

(75) Inventors: Fred Naraschkewitz, Buchholz Nordheide (DE); Marcus Eh, Holzminden (DE)

Correspondence Address: CONNOLLY BOVE LODGE & HUTZ, LLP, P O BOX 2207, WILMINGTON, DE, 19899, US

(73) Assignee: SYMRISE GmbH & Co. KG, Holzminden (DE)

(21) Appl. No.: 11/967,574

(22) Filed: Dec. 31, 2007

(30) Foreign Application Priority Data
Jan. 2, 2007 (EP) 07 100 018.6

(51) Int. Cl. A61K 8/33; A61Q 13/00

(52) U.S. Cl. 512/22

(57) ABSTRACT

A mixture is described which comprises or consists of 7.5 to 27.5 parts by weight of 3-(4-methylcyclohex-3-enyl)butyraldehyde (limonenal) and 92.5 to 72.5 parts by weight of 2,6-dimethyl-7-octen-2-ol (dihydromyrcenol), the sum of the parts by weight of 3-(4-methylcyclohex-3-enyl)butyraldehyde and 2,6-dimethyl-7-octen-2-ol being 100.

### RELATED APPLICATIONS

This application claims benefit of European application 07 100 018.6 filed Jan. 2, 2007.

According to a first feature, the present invention relates to mixtures comprising or consisting of specific amounts of 3-(4-methylcyclohex-3-enyl)butyraldehyde (limonenal) and 2,6-dimethyl-7-octen-2-ol (dihydromyrcenol). A further feature relates to corresponding products comprising mixtures according to the invention, said products preferably containing surfactants.

The invention further relates to the use of limonenal as a means of intensifying the citrus, bergamot-like top note and the naturalness of the odour of dihydromyrcenol.

The invention further relates to a method (i) of intensifying the citrus, bergamot-like top note and the naturalness of the odour of dihydromyrcenol, and to a method of preparing a mixture according to the invention that imitates or imparts the odour of bergamot oil.

Finally, the invention further relates to a method of providing (a) hair or (b) textile fibres with the odour of bergamot oil.

3-(4-Methylcyclohex-3-enyl)butyraldehyde (limonenal) is already known from the state of the art:

U.S. Pat. No. 2,584,539 describes the hydroformylation reaction of limonene to 3-(4-methylcyclohex-3-enyl)butyraldehyde. The odour of 3-(4-methylcyclohex-3-enyl)butyraldehyde is described as pleasant and long-lasting; limonenal is said to be used in a mixture with or as a substitute for hydroxycitronellal in perfume mixtures. It should be noted in this regard that hydroxycitronellal has a flowery odour.

U.S. Pat. No. 2,710,825 describes the preparation of 3-(4-methylcyclohex-3-enyl)butyraldehyde, inter alia, from limonene. The odour of 3-(4-methylcyclohex-3-enyl)butyraldehyde is described as stronger and longer-lasting than that of citral and it is maintained that the butyraldehyde (limonenal) additionally imparts a green or fresh note.

EP 011 272 A3 describes the preparation of aldehydes by the hydroformylation of olefins. Inter alia, the preparation of 3-(4-methylcyclohex-3-enyl)butyraldehyde by the hydroformylation of limonene is described. The odour of 3-(4-methylcyclohex-3-enyl)butyraldehyde is reported to be an agrumen and rhubarb note. It is also mentioned that this compound can be used not only in fine perfumery but also e.g. for the perfuming of soaps, cleaning agents and detergents or fabric softeners.

In our own experiments 3-(4-methylcyclohex-3-enyl)butyraldehyde (limonenal) was found to have a very intense, modern, fresh, green, aidehydic, somewhat fatty and citrus-like odour coupled with flowery-fruity notes. The green citrus-like odour is reminiscent of the peel of citrus fruits, especially lemons and limes.

Dihydromyrcenol is a common constituent of perfume compositions; it is used in particular in modern perfume oils to imitate (mimic) the odour of bergamot oil. Dihydromyrcenol is a colourless liquid which (like bergamot oil) has a fresh citrus-like odour and a lavender note. However, dihydromyrcenol does not impart the olfactorily complex impression of natural ethereal bergamot oil that is frequently preferred in the perfume industry, especially for classic perfume oils.

In practice, therefore, the odour of bergamot oil is alternatively mimicked very commonly by mixtures comprising linalyl acetate and linalool. These two compounds are the main constituents of natural bergamot oil. Bergamot oil is said in practice to be imitated olfactorily, i.e. replaced by synthetic fragrance mixtures, for a number of reasons:

Natural bergamot oil is only produced in small amounts and is therefore comparatively expensive; the bergamot fruit is only cultivated commercially along a narrow strip of the Calabrian coast (Italy).

The olfactory note of natural bergamot oil is subject to natural variations, but the perfume industry demands olfactory notes that are categorically reproducible.

Because of the presence of dihydrocumin alcohol (another constituent of bergamot oil), natural bergamot oil tends to discolour during storage.

Because of the presence of linalool (main constituent, see above), natural bergamot oil tends to sensitize the skin, cf. linalool, especially guideline 2003/15 EC and SÖFW-Journal, 130, 4-2004, pages 58 to 62.

Because of the allergenic properties of linalool, which is the main constituent occurring in bergamot oil, mixtures based on linalyl acetate and linalool, which are used industrially to imitate the odour of bergamot oil, are also increasingly becoming problematic.

Dihydromyrcenol, which, as already mentioned above, is used as an alternative substance for imitating the odour of bergamot oil, has not yet been able to assert itself in practical perfumery as a means of producing a bergamot oil odour, against mixtures based on linalyl acetate and linalool, even though it has advantageous properties and, in particular, neither tends to discolour nor has a sensitizing (allergenic) action.

The primary object of the present invention was therefore to provide a fragrance or fragrance mixture (perfume composition) that imparts or (together with other olfactory notes) comprises the odour of bergamot oil. Preferably, the fragrance to be provided or the fragrance mixture to be provided should mimic (imitate) the olfactorily complex impression of a natural ethereal bergamot oil.

This primary object is achieved according to the invention by a mixture comprising or consisting of

7.5 to 27.5 parts by weight of 3-(4-methylcyclohex-3-enyl)butyraldehyde (limonenal) and 92.5 to 72.5 parts by weight of 2,6-dimethyl-7-octen-2-ol (dihydromyrcenol), wherein the sum of the parts by weight of 3-(4-methylcyclohex-3-enyl)butyraldehyde and 2,6-dimethyl-7-octen-2-ol is 100.

The compounds limonenal and dihydromyrcenol contained in perfume compositions according to the invention can each be present as pure enantiomers or as mixtures of these enantiomers; limonenal is preferably used as an enantiomer mixture.

Preferably, the mixture according to the invention comprises or consists of

12.5 to 20 parts by weight of 3-(4-methylcyclohex-3-enyl)butyraldehyde (limonenal) and 87.5 to 80 parts by weight of 2,6-dimethyl-7-octen-2-ol (dihydromyrcenol).

In our own experiments on mixtures consisting of limonenal and dihydromyrcenol, it has been found that limonenal added to dihydromyrcenol in a proportion of 7.5 to 27.5 wt. %, preferably of 12.5 to 20 wt. % and very particularly preferably of 13 to 17 wt. %, based on the total amount of dihydromyrcenol and limonenal, results in a surprising intensification of the citrus, bergamot-like top note of the dihydromyrcenol and at the same time in an increase in the naturalness of the odour. In particular, in the preferred ranges (12.5 to 20 parts by weight, preferably 13 to 17 parts by weight), the addition of limonenal to dihydromyrcenol produces the olfactorily complex impression of a natural ethereal bergamot oil. Advantageously, moreover, when the parts by weight are adjusted to said values, the fatty note of limonenal (clearly perceived in a sensory analysis of pure limonenal), which is regarded as rather disadvantageous, is no longer perceptible.

The Table below contains information on odour descriptions of pure dihydromyrcenol (no. 1) and pure limonenal (no. 2) and of selected mixtures of dihydromyrcenol and limonenal (no. 3, 4, 5 and 6).

| | Proportion of | | |
|---|---|---|---|
| | dihydro- | Proportion of | |
| No. | myrcenol | limonenal | Odour description |
| 1 | 100% | 0% | Fresh, citrus, flowery |
| 2 | 0% | 100% | Fresh, citrus, green, aldehydic, |
| | | | somewhat fatty with flowery |
| | | | nuances |
| 3 | 75% | 25% | Fresh, citrus with a bergamot-like |
| | | | naturalness, aldehydic |
| 4 | 85% | 15% | Very attractive, fresh and intense |
| | | | citrus-like odour with very |
| | | | pronounced naturalness, closely |
| | | | approximating to the odour of |
| | | | bergamot oil. The naturalness of |
| | | | the odour is supported by the |
| | | | nuanced flowery aspects. |
| 5 | 90% | 10% | Fresh, natural citrus note with |
| | | | flowery, bergamot-like aspects |
| 6 | 95% | 5% | Fresh, somewhat metallic citrus |
| | | | note, without emanation |

It is recognized that, especially when using 10 or 15 wt. % of limonenal (based on the total amount of dihydromyrcenol and limonenal in the mixture), a particular naturalness has been detected. Overall, it is advantageous to have 12.5 to 20 parts by weight of limonenal together with 87.5 to 80 parts by weight of dihydromyrcenol in a mixture according to the invention (as stated above).

Preferred mixtures according to the invention are perfume compositions. Such perfume compositions preferably comprise or consist of

3-(4-methylcyclohex-3-enyl)butyraldehyde (limonenal), 2,6-dimethyl-7-octen-2-ol (dihydromyrcenol) and one, several or all other constituents selected from the group consisting of

fragrances, preferably in an amount of 100 parts by weight or more, and

fixatives.

The simultaneous presence of one or more other fragrances and fixatives in a perfume composition according to the invention is preferred. Apart from limonenal and dihydromyrcenol, preferred perfume compositions comprise an equal or higher proportion by weight of one or more other fragrances.

The use of a fixative in a preferred mixture (perfume composition) according to the invention increases the lingering power of the fragrances used, whether it be by lowering their vapour pressure or by intensifying the odour of the (other) fragrances (e.g. lowering the threshold value).

One particularly preferred mixture according to the invention (preferably a mixture according to the invention which is a perfume composition) comprises a total amount of 3-(4-methylcyclohex-3-enyl)butyraldehyde (limonenal) and 2,6-dimethyl-7-octen-2-ol (dihydromyrcenol) that imparts, intensifies or modifies a bergamot olfactory note.

Preferred mixtures (especially perfume compositions) according to the invention comprise no dihydrocumin alcohol and/or no linalool and/or no 3-(4-methylcyclohexyl)butyraldehyde. See above for the disadvantages associated with the use of dihydrocumin alcohol and/or linalool. 3-(4-Methylcyclohexyl)butyraldehyde is an unwanted by-product in the preparation of limonenal from limonene.

Mixtures according to the invention and especially perfume compositions according to the invention can be incorporated into a number of products, or applied to such products, with outstanding results. The invention therefore further relates to products comprising a mixture (especially perfume composition) according to the invention.

Mixtures (especially perfume compositions) according to the invention are particularly suitable for use in products containing surfactants. The invention therefore further relates to surfactant-containing products comprising a mixture (preferably perfume composition) according to the invention. There is a constant demand for fragrance mixtures with a citrus top note and pronounced naturalness, said fragrance mixtures preferably imitating the odour of bergamot oil, especially for the perfuming of formulations containing surfactants, e.g. cleaning agents.

In particular, if products containing surfactants are provided for the treatment of hair or textile fibres, another important application technology requirement is their substantivity towards or retention on the substrate, i.e. especially hair or textile fibres. The meaning of substantivity and retention is explained in detail e.g. in EP 1 201 738 Al, cf. sections [0004]-[0005], In general, therefore, fragrances with a high substantivity and/or retention are also sought after.

The mixtures (especially perfume compositions) according to the invention are particularly suitable for use in surfactant-containing products which satisfy the above-mentioned requirements.

A product according to the invention (preferably containing surfactants) is preferably one of the following:

an acidic, alkaline or neutral cleaning agent, preferably selected from the group consisting of all-purpose cleaners, floor cleaners, window cleaners, dishwashing detergent, bath and sanitaryware cleaners, scouring cream, solid and liquid WC cleaners, carpet cleaning powders and foams, liquid detergents, powder detergents, fabric preconditioners like bleach, soaker and stain removers, fabric softeners, washing soaps, washing tablets, disinfectants and surface disinfectants;

an air improver in liquid form, in gel form, applied to a solid carrier or in the form of an aerosol spray;

a wax or a polish, preferably selected from the group consisting of furniture polishes, floor waxes and shoe polishes; or

a body care product, preferably selected from the group consisting of solid and liquid soaps, shower gels, shampoos, shaving soaps, shaving foams, bath oils, cosmetic emulsions of the oil-in-water, water-in-oil and water-in-oil-in-water type, e.g. skin creams and lotions, face creams and lotions, sun creams and lotions, after-sun creams and lotions, hand creams and lotions, foot creams and lotions, depilatory creams and lotions, after-shave creams and lotions and tanning creams and lotions, hair care products, e.g. hair sprays, hair gels, hair lotions, hair rinses, permanent and semipermanent hair dyes, hair styling products like cold waving and straightening products, hair tonics, and hair creams and lotions, deodorants and antiperspirants, e.g. armpit sprays, roll-ons, deodorant sticks and deodorant creams, and decorative cosmetic products.

Ingredients with which the mixtures according to the invention can be combined are e.g.:

preservatives, abrasives, anti-acne agents, agents for combating skin ageing, antibacterial agents, anticellulite agents, antidandruff agents, anti-inflammatories, agents for preventing irritation, agents for inhibiting irritation, antimicrobial agents, antioxidants, astringents, perspiration-inhibiting agents, antiseptics, antistatics, binders, buffers, carriers, chelating agents, cell stimulants, cleansing agents, nurturing agents, depilatories, surface-active substances, deodorants, antiperspirants, softeners, emulsifiers, enzymes, ethereal oils, fibres, film-forming agents, fixatives, foaming agents, foam stabilizers, antifoams, foam boosters, fungicides, gelling agents, gel-forming agents, hair care agents, hair shaping agents, hair straightening agents, moisture-bestowing agents, moisturizing substances, humectants, bleaching agents, starching agents, stain-removing agents, optical brighteners, impregnating agents, dirt repellents, friction-reducing agents, lubricants, moisturizing creams, ointments, opacifiers, plasticizers, hiding agents, polish, lustring agents, polymers, powders, proteins, superfatting agents, gently scouring agents, silicones, skin soothing agents, skin cleansers, skin nurturing agents, skin healing agents, skin lightening agents, skin protecting agents, skin softening agents, cooling agents, skin cooling agents, warming agents, skin warming agents, stabilizers, UV absorbers, UV filters, detergents, fabric softeners, suspending agents, skin tanning agents, thickeners, vitamins, oils, waxes, fats, phospholipids, saturated fatty acids, monounsaturated or polyunsaturated fatty acids, α-hydroxy acids, polyhydroxy fatty acids, liquefying agents, colourants, colour protecting agents, pigments, anticorrosives, flavourings, taste-providing agents, fragrances, polyols, surfactants, electrolytes, organic solvents or silicone derivatives.

The present invention further relates to a method of intensifying the citrus, bergamot-like top note and the naturalness of the odour of 2,6-dimethyl-7-octen-2-ol (dihydromyrcenol). The invention further relates to a method of preparing a mixture (especially perfume composition) according to the invention that imitates or imparts the odour of bergamot oil, preferably a mixture designated above as preferred. The methods according to the invention comprise the following steps:

providing 3-(4-methylcyclohex-3-enyl)butyraldehyde (limonenal) and 2,6-dimethyl-7-octen-2-ol (dihydromyrcenol), and

mixing of

7.5 to 27.5 parts by weight of 3-(4-methylcyclohex-3-enyl)butyraldehyde (limonenal) and 92.5 to 72.5 parts by weight of 2,6-dimethyl-7-octen-2-ol (dihydromyrcenol), preferably 12.5 to 20 parts by weight of 3-(4-methylcyclohex-3-enyl)butyraldehyde (limonenal) and 87.5 to 80 parts by weight of 2,6-dimethyl-7-octen-2-ol (dihydromyrcenol), wherein the sum of the parts by weight of 3-(4-methylcyclohex-3-enyl)butyraldehyde and 2,6-dimethyl-7-octen-2-ol is 100.

The present invention further relates to the use of 3-(4-methylcyclohex-3-enyl)butyraldehyde (limonenal) as a means of intensifying the citrus, bergamot-like top note and the naturalness of the odour of 2,6-dimethyl-7-octen-2-ol (dihydromyrcenol).

A mixture according to the invention comprising or consisting of

7.5 to 27.5 parts by weight of 3-(4-methylcyclohex-3-enyl)butyraldehyde (limonenal) and 92.5 to 72.5 parts by weight of 2,6-dimethyl-7-octen-2-ol (dihydromyrcenol), preferably 12.5 to 20 parts by weight of 3-(4-methylcyclohex-3-enyl)butyraldehyde (limonenal) and 87.5 to 80 parts by weight of 2,6-dimethyl-7-octen-2-ol (dihydromyrcenol), can be used especially as a fragrance mixture for imitating the odour of bergamot oil (preferably for imitating the olfactorily complex impression of a natural ethereal bergamot oil).

The invention further relates to the use of a mixture (especially perfume composition) according to the invention

for imitating or imparting the odour of bergamot oil, or

as a means of providing (a) hair or (b) textile fibres with the odour of bergamot oil, preferably with the complex olfactory impression of a natural ethereal bergamot oil.

Preferably, within the framework of the use according to the invention, the mixture (preferably perfume composition) to be used is employed as a constituent of a product according to the invention.

Finally, the invention further relates to a method of providing (a) hair or (b) textile fibres with the odour of bergamot oil, preferably with the complex olfactory impression of a natural ethereal bergamot oil. Such a method comprises the following steps:

providing a mixture (preferably a mixture designated above as preferred) according to the invention or a product (preferably a product designated above as preferred) according to the invention, and

application of said mixture or said product to the hair or the textile fibres.

One particularly preferred mixture according to the invention, which is particularly suitable for the uses according to the invention or the corresponding methods, is a solution comprising

(a) water,

(b) 7.5 to 27.5 parts by weight of 3-(4-methylcyclohex-3-enyl)butyraldehyde (limonenal) and 92.5 to 72.5 parts by weight of 2,6-dimethyl-7-octen-2-ol (dihydromyrcenol), preferably 12.5 to 20 parts by weight of 3-(4-methylcyclohex-3-enyl)butyraldehyde (limonenal) and 87.5 to 80 parts by weight of 2,6-dimethyl-7-octen-2-ol (dihydromyrcenol), and

(c) one or more surfactants,

the concentration of limonenal in the solution being in the range from 10⁷ to 10⁻¹ wt. %.

Other fragrances and/or miscellaneous conventional additives may be present.

The documents of the state of the art acknowledged above make no reference to the particular olfactory effects of 3-(4-methylcyclohex-3-enyl)butyraldehyde (limonenal) in combination with dihydromyrcenol, especially in the context of preferably aqueous products containing surfactants. Surprisingly, through the combined use of limonenal and dihydromyrcenol, the uses, methods, mixtures (especially perfume compositions) and products according to the invention achieve an olfactorily complex impression that can otherwise only be achieved by natural ethereal bergamot oils.

The mixture (preferably perfume composition) according to the invention can be used in a large number of products The pH of an aqueous formulation containing the mixture (preferably perfume composition) according to the invention is not critical as both dihydromyrcenol and limonenal are stable in weakly acidic as well as basic media. It has been found, however, that limonenal (especially in applications/formulations with a basic pH) has a particularly pronounced blooming and effects an increase in the blooming of dihydromyrcenol, so the pH of aqueous products according to the invention, preferably containing surfactants, is preferably in the range from 8to 11.

Examples of fragrances with which limonenal and dihydromyrcenol can advantageously be combined can be found e.g. in S. Arctander, Perfume and Flavor Materials, vol. I and II, Montclair, N.J., 1969, Selbstverlag, or K. Bauer, D. Garbe and H. Surburg, Common Fragrance and Flavor Materials, 4th ed., Wiley-VCH, Weinheim 2001.

The amount of 3-(4-methylcyclohex-3-enyl)butyraldehyde (limonenal) used in mixtures (especially perfume compositions) according to the invention is preferably 0.1 to 10 wt. %, particularly preferably 0.5 to 5 wt. %, based on the total mixture. The amount of dihydromyrcenol used is obtained from the above data on mixtures according to the invention, the data on preferred mixtures also being applicable in this regard.

Mixtures (especially perfume compositions) according to the invention that are based on perfume oils containing limonenal and dihydromyrcenol can be used in concentrated form, in solutions or in the modified forms described below for the perfuming of e.g. acidic, alkaline and neutral cleaning agents such as carpet cleaning powders and foams, liquid detergents, powder detergents, fabric preconditioners like bleach, soaker and stain remover, fabric softeners, washing soaps, washing tablets, body care products such as solid and liquid soaps, shower gels, shampoos, cosmetic emulsions of the oil-in-water, water-in-oil and water-in-oil-in-water type, and hair care products such as hairsprays, hair gels, strengthening hair lotions, hair rinses, permanent and semipermanent hair dyes, hair styling products like cold waving and straightening products, hair tonics, and hair creams and lotions.

Perfume oils containing the mixtures (especially perfume compositions) according to the invention can be used in perfumed products in liquid form, either undiluted or diluted with a solvent. Examples of suitable solvents for this purpose are ethanol, isopropanol, diethylene glycol monoethyl ether, glycerol, propylene glycol, 1,2-butylene glycol, dipropylene glycol, diethyl phthalate, triethyl citrate, isopropyl myristate, etc.

Perfume oils containing the mixtures (especially perfume compositions) according to the invention can also be adsorbed on a carrier to ensure both a fine distribution of the fragrances in the product and a controlled release when applied. Such carriers can be porous inorganic materials such as light sulfate, silica gels, zeolites, gypsums, clays, clay granules, gas concrete, etc., or organic materials such as woods and cellulose-based substances.

Perfume oils containing the mixtures (especially perfume compositions) according to the invention can also be microencapsulated or spray-dried or in the form of inclusion complexes or extrusion products, and can be added in this form to the product to be perfumed.

Optionally, the properties of the perfume oils modified in this way can be further optimized, in respect of a more specific perfume release, by coating with suitable materials; waxy plastics, e.g. polyvinyl alcohol, are preferably used for this purpose.

Microencapsulation of the perfume oils can be effected e.g. by the so-called coacervation process with the aid of capsule materials made e.g. of polyurethane-like substances or soft gelatin. Spray-dried perfume oils can be prepared e.g. by spray drying an emulsion or dispersion containing the perfume oil, it being possible for modified starches, proteins, dextrins and vegetable gums to be used as carriers. Inclusion complexes can be prepared e.g. by introducing dispersions of the perfume oil and cyclodextrins or urea derivatives into a suitable solvent, e.g. water. Extrusion products can be prepared by melting the perfume oils with a suitable waxy substance and by extrusion with subsequent solidification, optionally in a suitable solvent, e.g. isopropanol.

Preferred products which can be used within the framework of the present invention are (a) perfume oil mixtures for products (especially formulations) containing surfactants, e.g. cleaning agents, detergents, fabric softeners and body care products, and (b) the corresponding products (especially formulations) themselves which contain surfactants.

The surfactant-containing products (especially formulations) which can be used within the framework of the present invention generally include substances from the class of anionic surfactants, e.g. carboxylates, sulfates, sulfonates and phosphates, cationic surfactants, e.g. quaternary ammonium salts, amphoteric surfactants, e.g. betaines, and non-ionic surfactants, e.g. ethoxylates and propoxylates.

Preferred anionic surfactants are sulfates and sulfonates. Preferred sulfates are those having 12 to 18 carbon atoms and a degree of ethoxylation of 1 to at most 5. Sodium laurylethersulfate, preferably having a mean degree of ethoxylation of 2 to 4, is particularly preferred.

Particularly preferred sulfonates are linear sodium alkylbenzenesulfonates having an average of approx. 12 carbon atoms in the alkyl chain, said alkyl chains consisting of homologous radicals having 10 to 14 carbon atoms (“dodecylbenzenesulfonate”).

Preferred compounds from the group of non-ionic surfactants are ethoxylated fatty alcohols obtained by the ethoxylation of alcohols having 12 to 18 carbon atoms (fatty alcohol ethoxylates having 12 to 18 C atoms). The degree of ethoxylation here can vary within wide limits, but particularly preferred products are those having an average degree of ethoxylation of 5 to 10 or, in particular, 7 mol of added ethylene oxide per mol of fatty alcohol.

Particularly preferred betaines are those of the acid amide type having the structure shown:

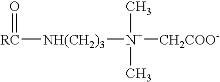

A preferred radical RC═O is the coconut oil fatty acid cut in which lauric acid is the main constituent at 45-50%.

In combination with selected surfactants, the favourable properties of the mixture (especially perfume composition) according to the invention based on limonenal and dihydromyrcenol are surprisingly pronounced. A corresponding surfactant-containing product according to the invention preferably comprises, in addition to limonenal and dihydromyrcenol (in the proportions by weight according to the invention), one or more surfactants selected from the group consisting of:

linear alkylbenzenesulfonates (especially those mentioned above, e.g. linear sodium alkylbenzenesulfonates),

fatty alcohol ethoxylates having 12-18 C atoms (especially those mentioned above, e.g. those having the degree of ethoxylation identified above as preferred),

laurylethersulfates (especially those mentioned above, e.g. the sodium laurylethersulfate mentioned above) and

betaines (especially those mentioned above, e.g. betaines of the acid amide type having the structure shown above).

Linear alkylbenzenesulfonates and fatty alcohol ethoxylates having 12-18 C atoms are preferably used together with one another here, especially in heavy-duty detergent powders.

Likewise, laurylethersulfates (especially the sodium laurylethersulfate mentioned above) and betaines (especially those of the acid amide type having the structure shown above) are preferably used together with one another, especially in light-duty detergents, shampoos and shower gels.

The concentration of surface-active substances in the surfactant-containing products according to the invention is not normally critical. Preferred concentrations depend on the type of surfactant and the particular application. For example, they can be less than 1 wt. % in special bleach products, but greater than 99 wt. % in soaps or washing powder.

Particular combinations and concentrations are preferred in surfactant-containing products according to the invention for particular fields of application. Thus, preferred mixtures according to the invention (detergent formulations) are those in which the proportion of linear alkylbenzenesulfonates is in the range from 7 to 10 wt. % and/or the proportion of fatty alcohol ethoxylates having 12-18 C atoms is in the range from 3 to 6 wt. %, based in each case on the total weight of the mixture. Other preferred mixtures according to the invention (formulations for light-duty detergents, shampoos and shower gels) are those in which the proportion of sodium laurylethersulfate is in the range from 7 to 13 wt. % and/or the proportion of betaine (especially betaine of the acid amide type having the structure shown above) is in the range from 1 to 3 wt. %, based in each case on the total weight of the mixture.

When surfactant-containing products according to the invention are applied, the substantivity of the 3-(4-methylcyclohex-3-enyl)butyraldehyde (limonenal) and dihydromyrcenol on hair and textile fibres is so pronounced as to give the impression that although the surfactants present initially bring the limonenal and the dihydromyrcenol into the aqueous phase, these compounds, in the presence of hair or textile fibres, are forced out of the aqueous phase and onto the hair or textile fibre. However, there is currently no scientific explanation for this observation.

The surfactant-containing products containing limonenal and dihydromyrcenol exhibit a surprisingly high substantivity or retention towards or on hair, wool, cotton and other textile fibres.

The invention further relates to the use of limonenal for increasing the substantivity of dihydromyrcenol.

The Examples which follow serve to illustrate the invention:

### EXAMPLES 1-3

Preliminary remark:

In Examples 1-3 below, the properties of “substantivity” and “blooming” were evaluated by a panel of experts (8-12 persons).

For the determination of substantivity, unperfumed fabric softener, shampoo or washing powder was perfumed with a perfume composition according to the invention consisting of limonenal and dihydromyrcenol in a weight ratio of 15:85 or with the conventional concentration of dihydromyrcenol (substance used for comparison). The olfactory assessment was made using a 7-point scale with values from 0 (no odour) to 6 (very strong odour).

For the assessment of blooming, an aqueous solution containing a low concentration of surfactants was treated with 0.1% of said perfume composition according to the invention consisting of limonenal and dihydromyrcenol or with dihydromyrcenol (substance used for comparison). The olfactory assessment was made using a 7-point scale with values from 0 (no odour) to 6 (very strong odour).

The “stability” property was determined on the basis of the residual amount (in %) of the test fragrances after a storage period of 1 month (1 M) or 2 months (2 M) at a constant temperature of 40° C. in the respective ready-to-use formulations (i.e. after incorporation of the respective fragrance into the appropriate base) of the following Examples.

### Determination of the Substantivity (Examples 1-3) and Stability (Examples 1 and 3)

### EXAMPLE 1

### Shampoo

The substance to be evaluated was incorporated as a 50 wt. % solution in diethyl phthalate, in a dose of 0.6 wt. %, into a shampoo base of the following composition:

| | |
|---|---|
| Sodium laurylethersulfate | 12% |
| (e.g. Texapon NSO from Cognis Deutschland GmbH) | |
| Cocamidopropylbetaine | 2% |
| (e.g. Dehyton K from Cognis Deutschland GmbH) | |
| Sodium chloride | 1.4% |
| Citric acid | 1.3% |
| Phenoxyethanol, methyl-, ethyl-, butyl- and | 0.5% |
| propylparaben | |
| Water | 82.8% |

The pH of the shampoo base was about 6. This is used to prepare 100 ml of a 20 wt. % aqueous shampoo solution (as an Example of a solution according to the invention). Two swatches of hair are washed together for 2 minutes in this shampoo solution and then rinsed for 20 seconds under lukewarm running water. One swatch is packed wet in aluminium foil and the second swatch is dried with a hair dryer. Both swatches are assessed by a panel for their olfactory properties.

| | Evaluation of | | |
|---|---|---|---|
| | substantivity | | |
| Substance to be evaluated | wet | dry | Stability |
| Mixture of limonenal and dihydro- | 3.0 | 2.2 | 1 M: 100% |
| myrcenol in a weight ratio of | | | 2 M: 95% |
| 15:85 (according to the invention) | | | |
| Dihydromyrcenol | 2.5 | 0.9 | 1 M: 96% |
| (comparison) | | | 2 M: 88% |

The use of limonenal in the mixture according to the invention led to an increase in the substantivity of the dihydromyrcenol.

### EXAMPLE 2

### Fabric Softener

The substance to be evaluated is incorporated as a 50 wt. % solution in diethyl phthalate, in a dose of 0.5 wt. %, into a fabric softener base of the following composition:

| | |
|---|---|
| Quaternary ammonium methosulfate | 5.5% |
| (Esterquat), approx. 90% | |
| (e.g. Rewoquat WE 18 from Witco Surfactants GmbH) | |
| Alkyldimethylbenzylammonium chloride, | 0.2% |
| approx. 50% | |
| (e.g. Preventol R50 from Bayer AG) | |
| Dye solution, approx. 1% | 0.3% |
| Water | 94.0% |

The pH of the fabric softener base was in the range from 2 to 3. Two cloths are rinsed for 30 minutes at 20° C. with 370 g of a 1% aqueous fabric softener solution prepared from the base (as an Example of a solution according to the invention) in a Linetest machine running the fabric softener programme. The cloths are wrung out and then spun for 20 seconds. One cloth is sealed up wet and one is hung up to dry. Both cloths are then assessed by a panel for their olfactory properties.

| | Evaluation of substantivity | |
|---|---|---|
| Substance to be evaluated | wet | dry |
| Mixture of limonenal and dihydromyrcenol | 2.8 | 1.8 |
| in a weight ratio of 15:85 (according to the | | |
| invention) | | |
| Dihydromyrcenol | 1.6 | 0.7 |
| (comparison) | | |

The use of limonenal in the mixture according to the invention led to an increase in the substantivity of the dihydromyrcenol.

### EXAMPLE 3

### Washing Powder

The substance to be evaluated is incorporated as a 50 wt. % solution in diethyl phthalate, in a i5 dose of 0.4 wt. %, into a washing powder base of the following formulation:

| | |
|---|---|
| Linear Na alkylbenzenesulfonate | 8.8% |
| Ethoxylated C12-18 fatty alcohol (7 EO) | 4.7% |
| Na soap | 3.2% |
| Antifoam | 3.9% |
| (Dow Corning ® 2-4248S Powdered Antifoam, | |
| silicone oil on zeolite as carrier) | |
| Zeolite 4A | 28.3% |
| Na carbonate | 11.6% |
| Na salt of an acrylic acid/maleic acid | 2.4% |
| copolymer | |
| (Sokalan CP5) | |
| Na silicate | 3.0% |
| Carboxymethyl cellulose | 1.2% |
| Dequest 2066 | 2.8% |
| ([[(phosphonomethyl)imino]bis[(ethylene- | |
| nitrilo)bis(methylene)]]tetrakisphosphonic | |
| acid, sodium salt) | |
| Optical brightener | 0.2% |
| Na sulfate | 6.5% |
| Protease | 0.4% |
| Sodium perborate tetrahydrate | 22.0% |
| TAED | 1.0% |

Two cloths are washed for 45 minutes at 60° C. with 370 g of a 1% aqueous washing powder liquor prepared from the base (as an Example of a surfactant-containing solution according to the invention, the pH of the washing powder liquor being well in the basic range) in a Linetest machine running the main washing cycle. The cloths are first rinsed for 5 minutes with cold water, wrung out and then spun for 20 seconds. One cloth is sealed up wet and one is hung up to dry. Both cloths are then assessed by a panel for their olfactory properties.

| | Evaluation of | | |
|---|---|---|---|
| | substantivity | | |
| Substance to be evaluated | wet | dry | Stability |
| Mixture of limonenal and dihydromyrcenol | 2.5 | 1.8 | 1 M: 99% |
| in a weight ratio of 15:85 (according to the | | | 2 M: 98% |
| invention) | | | |
| Dihydromyrcenol | 1.3 | 0.6 | 1 M: 98% |
| (comparison) | | | 2 M: 98% |

The use of limonenal in the mixture according to the invention led to an increase in the substantivity of the dihydromyrcenol.

### Determination of the Blooming (Example 4)

### EXAMPLE 4

The blooming of the shampoo solution of Example 1, the fabric softener solution of Example 2 and the washing powder liquor of Example 3 was assessed in each case by a panel on a scale of 0- 6 from an open 250 ml glass beaker.

| | Shampoo solution |
|---|---|
| Substance to be evaluated | (Example 1) |
| Mixture of limonenal and dihydromyrcenol | 4.0 |
| in a weight ratio of 15:85 (according to | |
| the invention) | |
| Dihydromyrcenol | 3.4 |
| (comparison) | |

| | Fabric softener |
|---|---|
| Substance to be evaluated | solution (Example 2) |
| Mixture of limonenal and dihydromyrcenol | 4.4 |
| in a weight ratio of 15:85 (according to | |
| the invention) | |
| Dihydromyrcenol | 3.5 |
| (comparison) | |

| | Washing powder |
|---|---|
| Substance to be evaluated | liquor (Example 3) |
| Mixture of limonenal and dihydromyrcenol | 3.3 |
| in a weight ratio of 15:85 (according to | |
| the invention) | |
| Dihydromyrcenol | 2.7 |
| (comparison) | |

The use of limonenal in the mixture according to the invention led in each case to an increase in the blooming of the dihydromyrcenol.

Perfume Compositions

Abbreviations used:

DPG: dipropylene glycol; IPM: isopropyl myristate

### Example 5: Perfume Composition/Perfume Oil

### EXAMPLE 5

Perfume oil, particularly suitable for incorporation into shampoo.

| | |
|---|---|
| Allylamyl glycolate | 4.00 |
| Calone 1951 | 1.00 |
| Canthoxal | 2.00 |
| Cassis 345B | 2.00 |
| Cedarleaf oil | 2.00 |
| Cedarwood oil | 5.00 |
| Cypress oil | 7.00 |
| Alpha-damascone | 0.50 |
| Eugenol | 8.00 |
| Evernyl | 0.50 |
| Fleursandol | 40.00 |
| Florazone | 5.00 |
| Galaxolide, 50% in DEP | 35.00 |
| Grapefruit base | 3.00 |
| Hedione | 288.0 |
| Helional | 60.00 |
| Cis-3-hexenol | 2.00 |
| Iso E Super | 50.00 |
| Lavender oil Abrialis Nat. | 15.00 |
| Lemon oil, winter, Italie | 20.00 |
| Ligustral | 2.00 |
| Lilial | 20.00 |
| Mandarin oil Super | 20.00 |
| Orange oil, Brazilian type | 15.00 |
| Patchouli oil, ”Entf. DM” | 10.00 |
| Rosemary oil, Spanish type | 5.00 |
| Sage oil, Dalmatian | 3.00 |
| Sandranol ® | 5.00 |
| Tonalide | 10.00 |
| Dihydromyrcenol | 306.00 |
| 3-(4-Methylcyclohex-3-enyl)butyraldehyde (limonenal) | 54.00 |
| TOTAL | 1000.00 |

Compared with a mixture containing 280 parts by weight of bergamot oil instead of a total of 280 parts by weight of dihydromyrcenol and 3-(4-methylcyclohex-3-enyl)butyraldehyde (limonenal), the above perfume oil containing dihydromyrcenol and limonenal had a noticeably more natural and tangier citrus top note; the top note was also found to have a better lingering power.

### Further Embodiments

A first embodiment of the present invention is a mixture comprising or consisting or consisting essentially of

7.5 to 27.5 parts by weight of 3-(4-methylcyclohex-3-enyl)butyraldehyde (limonenal) and 92.5 to 72.5 parts by weight of 2,6-dimethyl-7-octen-2-ol (dihydromyrcenol), wherein the sum of the parts by weight of 3-(4-methylcyclohex-3-enyl)butyraldehyde and 2,6-dimethyl-7-octen-2-ol is 100.

A second embodiment is a mixture according to the first embodiment comprising or consisting 1s or consisting essentially of 12.5 to 20 parts by weight of 3-(4-methylcyclohex-3-enyl)-butyraldehyde (limonenal) and 87.5 to 80 parts by weight of 2,6-dimethyl-7-octen-2-ol (dihydromyrcenol). A third embodiment is a mixture according to the first or second embodiment, wherein the mixture is a perfume composition comprising or consisting of 3-(4-methylcyclohex-3-enyl)butyraldehyde (limonenal), 2,6-dimethyl-7-octen-2-ol (dihydromyrcenol) and one, several or all other constituents selected from the group consisting of:

fragrances, preferably in an amount of 100 parts by weight or more, and

fixatives.

A fourth embodiment is a mixture according to one of the first through third embodiments comprising a total amount of 3-(4-methylcyclohex-3-enyl)butyraldehyde (limonenal) and 2,6-dimethyl-7-octen-2-ol (dihydromyrcenol) that imparts, intensifies or modifies a bergamot olfactory note. A fifth embodiment is a mixture according to one of the first through fourth embodiments that does not comprise dihydrocumin alcohol and/or linalool and/or 3-(4-methylcyclohexyl)butyraldehyde. A sixth embodiment is a product comprising a mixture according to one of the first through fifth embodiments. A seventh embodiment is a product according to the sixth embodiment, wherein the product is a surfactant-containing product. An eighth embodiment is a product according to the sixth or seventh embodiments, wherein the product is:

an acidic, alkaline or neutral cleaning agent, preferably selected from the group consisting of all-purpose cleaners, floor cleaners, window cleaners, dishwashing detergent, bath and sanitaryware cleaners, scouring cream, solid and liquid WC cleaners, carpet cleaning powders and foams, liquid detergents, powder detergents, fabric preconditioners like bleach, soaker and stain removers, fabric softeners, washing soaps, washing tablets, disinfectants and surface disinfectants;

an air improver in liquid form, in gel form or applied to a solid carrier or in the form of an aerosol spray;

a wax or a polish, preferably selected from the group consisting of furniture polishes, floor waxes and shoe polishes; or

a body care product, preferably selected from the group consisting of solid and liquid soaps, shower gels, shampoos, shaving soaps, shaving foams, bath oils, cosmetic emulsions of the oil-in-water, water-in-oil and water-in-oil-in-water type, hair care products, deodorants and antiperspirants, and decorative cosmetic products.

A ninth embodiment is the use of 3-(4-methylcyclohex-3-enyl)butyraldehyde (limonenal) as a means of intensifying the citrus, bergamot-like top note and the naturalness of the odour of 2,6-dimethyl-7-octen-2-ol (dihydromyrcenol). A tenth embodiment is the use of a mixture according to one of the first through fifth embodiments

for imitating or imparting the odour of bergamot oil, or

as a means of providing (a) hair or (b) textile fibres with the odour of bergamot oil.

An eleventh embodiment is the use according to the tenth embodiment wherein the mixture is used as a constituent of a product according to one of the sixth through eighth embodiments.

A twelfth embodiment is a method of

(i) intensifying the citrus, bergamot-like top note and the naturalness of the odour of 2,6-dimethyl-7-octen-2-ol (dihydromyrcenol), and/or

(ii) preparing a mixture according to one of the first through fifth embodiments that imitates or imparts the odour of bergamot oil, comprising the following steps:

providing 3-(4-methylcyclohex-3-enyl)butyraldehyde (limonenal) and 2,6-dimethyl-7-octen-2-ol (dihydromyrcenol), and

mixing of

7.5 to 27.5 parts by weight of 3-(4-methylcyclohex-3-enyl)butyraldehyde (limonenal) and 92.5 to 72.5 parts by weight of 2,6-dimethyl-7-octen-2-ol (dihydromyrcenol), preferably 12.5 to 20 parts by weight of 3-(4-methylcyclohex-3-enyl)butyraldehyde (limonenal) and 87.5 to 80 parts by weight of 2,6-dimethyl-7-octen-2-ol (dihydromyrcenol), wherein the sum of the parts by weight of 3-(4-methylcyclohex-3-enyl)butyraldehyde and 2,6-dimethyl-7-octen-2-ol is 100.

A thirteenth embodiment is a method of providing (p) hair or (b) textile fibres with the odour of bergamot oil, comprising the following steps:

providing a mixture according to one of the first through fifth embodiments or a product according to one of the sixth through eighth embodiments, and

applying the mixture or product to the hair or the textile fibres.

A fourteenth embodiment is the use of limonenal for increasing the substantivity and/or blooming of dihydromyrcenol.

1. A mixture comprising
7.5 to 27.5 parts by weight of 3-(4-methylcyclohex-3-enyl)butyraldehyde (limonenal) and 92.5 to 72.5 parts by weight of 2,6-dimethyl-7-octen-2-ol (dihydromyrcenol), wherein the sum of the parts by weight of 3-(4-methylcyclohex-3-enyl)butyraldehyde and 2,6-dimethyl-7-octen-2-ol is 100.

2. The mixture of claim 1, wherein the mixture consists of
7.5 to 27.5 parts by weight of 3-(4-methylcyclohex-3-enyl)butyraldehyde ( limonenal) and
92.5 to 72.5 parts by weight of 2,6-dimethyl-7-octen-2-ol (dihydromyrcenol), wherein the sum of the parts by weight of 3-(4-methylcyclohex-3-enyl)butyraldehyde and 2,6-dimethyl-7-octen-2-ol is 100.

3. The mixture of claim 1 comprising
12.5 to 20 parts by weight of 3-(4-methylcyclohex-3-enyl)butyraldehyde (limonenal) and 87.5 to 80 parts by weight of 2,6-dimethyl-7-octen-2-ol (dihydromyrcenol).

4. The mixture of claim 3 consisting of
12.5 to 20 parts by weight of 3-(4-methylcyclohex-3-enyl)butyraldehyde (limonenal) and 87.5 to 80 parts by weight of 2,6-dimethyl-7-octen-2-ol (dihydromyrcenol).

5. A perfume composition comprising the mixture of claim 1 and further comprising at least one constituent selected from the group consisting of:
fragrances and
fixatives.

6. The mixture of claim 1 comprising a total amount of 3-(4-methylcyclohex-3-enyl)-butyraldehyde (limonenal) and 2,6-dimethyl-7-octen-2-ol (dihydromyrcenol) that imparts, intensifies or modifies a bergamot olfactory note.

7. The mixture of claim 1 that does not comprise dihydrocumin alcohol and/or linalool and/or 3-(4-methylcyclohexyl)butyraldehyde.

8. A product comprising a mixture of claim 1.

9. The product of claim 8, wherein the product further comprises a surfactant.

10. The product of claim 8, wherein the product is:
an acidic, alkaline or neutral cleaning agent;
an air improver in liquid form, in gel form or applied to a solid carrier or in the form of an aerosol spray;
a wax or a polish; or
a body care product.

11. A method of intensifying the citrus, bergamot-like top note and the naturalness of the odour of 2,6-dimethyl-7-octen-2-ol (dihydromyrcenol) in a composition comprising comprising adding 3-(4-methylcyclohex-3-enyl)butyraldehyde (limonenal) to the composition.

12. A method of preparing a mixture according to claim 1 that imitates or imparts the odour of bergamot oil comprising the following steps:
providing 3-(4-methylcyclohex-3-enyl)butyraldehyde (limonenal) and 2,6-dimethyl-7-octen-2-ol (dihydromyrcenol), and
mixing together
7.5 to 27.5 parts by weight of 3-(4-methylcyclohex-3-enyl)butyraldehyde (limonenal) and 92.5 to 72.5 parts by weight of 2,6-dimethyl-7-octen-2-ol (dihydromyrcenol),
wherein the sum of the parts by weight of 3-(4-methylcyclohex-3-enyl)butyraldehyde and 2,6-dimethyl-7-octen-2-ol is 100.

13. The method of claim 12 comprising mixing together
12.5 to 20 parts by weight of 3-(4-methylcyclohex-3-enyl)butyraldehyde (limonenal) and 87.5 to 80 parts by weight of 2,6-dimethyl-7-octen-2-ol (dihydromyrcenol).

14. A method of providing (a) hair or (b) textile fibres with the odour of bergamot oil, comprising the following steps:
providing a mixture according to claim 1 and
applying the mixture to the hair or the textile fibres.

15. A method for increasing the substantivity and/or blooming of dihydromyrcenol in a composition comprising adding 3-(4-methylcyclohex-3-enyl) butyraldehyde (limonenal) to the composition in an amount sufficient such that the substantivity and/or blooming of dihydromyrcenol is increased.
